(19) United States
(12) Patent Application Publication
(10) Pub. No.: US 2025/0169774 A1
(43) Pub. Date: May 29, 2025

(54) SERVICE CRANES FOR MEDICAL SYSTEMS

(71) Applicant: RefleXion Medical, Inc., Hayward, CA (US)

(72) Inventors: William Roy KNAPP, Culver City, CA (US); Blake Hunt GADERLUND, Mountain View, CA (US)

(21) Appl. No.: 18/843,174

(22) PCT Filed: Mar. 1, 2023
(86) PCT No.: PCT/US2023/063491

§ 371 Date: Aug. 30, 2024

Related U.S. Application Data
(60) Provisional application No. 63/316,214, filed on Mar. 3, 2022.

(51) Int. Cl. A61B 6/04

(52) U.S. Cl. CPC A61B 6/0407

(57) ABSTRACT

A removable crane for servicing a medical system is provided. In a first implementation, the removable crane couples to a couch of the medical system, wherein upon securing the removable crane to the couch, a jib of the removable crane is positioned to enter a bore of a drum of the medical system upon a movement of the couch towards the drum. In a second implementation, the crane is permanently coupled to the gantry, such that the crane can be in a first configuration for use and a second stowed configuration.

### CROSS-REFERENCE TO RELATED APPLICATIONS

This application is a National Stage application under 35 U.S.C. § 371 of the International Application No. PCT/US2023/063491, filed Mar. 1, 2023, which claims priority to U.S. Provisional Patent Application No. 63/316,214 filed Mar. 3, 2022, which is incorporated by reference in its entirety.

### TECHNICAL FIELD

Disclosed herein are systems and methods for servicing a medical system and more particularly cranes for lifting components of a medical system.

### BACKGROUND

A medical system may have a bore that surrounds the patient treatment area. Example of such medical systems include radiotherapy systems (a radiotherapy system may have a radiation source such as a linear accelerator mounted so as to be movable around a patient support on which a patient can be placed for treatment), imaging systems (e.g., magnetic resonance imaging (MRI) system, computed tomography (CT) system, or positron emission tomography (PET) system), or a combination thereof. Such medical systems may include a radiation source and/or imaging system(s) that are movable around the bore. The patient support (e.g., a couch) is usually movable into and out of the bore of the medical system. One or more components of the medical system may be heavy and/or bulky. In many cases, when repairs or adjustments are needed to the system equipment (e.g., linear accelerator), the equipment needs to be lifted and moved using a lifting apparatus, as many of the constituent parts of the imaging or radiotherapy systems are large, bulky and/or too heavy to be lifted and accurately maneuvered manually in the limited space available in a treatment bunker. For example, a multi-leaf collimator which is used to collimate the radiation beam emitted by a linear accelerator could weigh 80 pounds or more, and PET arcs may weigh as much 250 pounds or more. A lifting apparatus, such as a crane would be helpful to help lift these component pieces for servicing, but space limitations of the treatment bunker may make it very difficult to maneuver within the treatment space, particularly when it is carrying a load.

Accordingly, there is a need for compact, easily deployable cranes that can maneuver within the limited maintenance space available of medical systems.

### SUMMARY

The present disclosure relates to devices and methods for servicing a medical system. One variation includes a removable crane that is configured to couple to a couch of the medical system. A method of using a couch-mounted removable crane may include securing the removable crane to the couch, moving the couch into the bore of a drum of a medical system, and positioning a jib of the removable crane within the bore as desired to engage and maneuver a component of the medical system.

One variation may include a removable crane installation system for servicing a medical system. The medical system may include a gantry, a drum attached to a front face of a gantry, the drum having a bore, and a couch capable of moving towards and away from the drum. The removable crane installation system may include a removable crane configured to couple to the couch via a couch coupling mechanism, the removable crane having a tower and a jib connected to the tower, wherein, upon securing the tower to the couch, the jib is positioned to enter the opening of the drum upon a movement of the couch towards the drum. In some variations, upon securing the removable crane to the couch, the jib of the removable crane may be substantially parallel to an axis of the bore. The removable crane may be configured to couple to the couch via a couch coupling mechanism attached to the couch. For example, the couch coupling mechanism may include a clamp attached to a distal end of the couch and/or the couch coupling mechanism may include a left clamp attached to a left side of the couch and a right clamp attached to a right side of the couch. The removable crane may include at least one extension element, and the couch coupling mechanism may include an opening configured to receive the extension element and latch to the extension element. The couch coupling mechanism may be configured to be attached at a distal end of the medical couch. Alternatively, or additionally, the couch coupling mechanism may be configured to be attached at a position of the couch located between a substantially middle position of the couch and a distal end of the couch. In some variations, the removable crane may be configured to execute at least one of a lateral or rotational motion relative to the couch. As an example, the couch coupling mechanism may include a first element attached to the couch and a second element attached to the removable crane, and the second element may be configured to move laterally relative to the first element. The first element may include one or more rails mounted on a platform configured to rotate in a plane parallel to a top surface of the couch, and the second element may include a set of wheels configured to move along the one or more rails. An attachment of the removable crane to the second element may allow the removable crane to rotate relative to the second element in a plane parallel to the top surface of the couch.

In some variation, a medical system may include a gantry, a drum attached to a front face of a gantry, the drum having a bore, and a couch capable of moving towards and away from the drum. The medical system may include at least one of a positron emission tomography (PET) device, and a computed tomography (CT) device. Some medical systems may comprise a drum that includes components located inside the bore. One variation of a removable crane installation system for servicing such a medical system may comprise a removable crane configured to couple to the couch via a couch coupling mechanism, the removable crane having a tower and a jib connected to the tower, where upon securing the tower to the couch, the jib may be positioned to enter the opening of the drum upon a movement of the couch towards the drum. The tower may be mounted substantially perpendicular to a top surface of the couch, and the jib may be positioned substantially perpendicular to the tower. The jib may be configured to have a size that allows the jib to be inserted into the bore while lifting a component inside the bore, such that the component does not touch any sides of the bore. In some variations, the couch coupling mechanism may be attached to the couch via a left clamp attached to a left side of the couch and a right clamp attached to a right side of the couch. Alternatively, or additionally, the couch coupling mechanism may include a groove, and the removable crane may include an extension element configured to slidably engage with the groove of the couch coupling mechanism. In some variations, the couch coupling mechanism may include an opening, and the removable crane may include an extension element configured to slidably engage with the opening of the couch coupling mechanism.

Another disclosed variation may include a removable crane installation system for servicing a medical system. The medical system comprises a couch, the couch including a couch patient platform having a platform base and a platform extension, where the platform extension is attached to the platform base via an attachment mechanism. The removable crane installation system may include a removable crane, configured to attach to the attachment mechanism of the platform base. A jib of the removable crane may be configured to be moved into a bore of a drum of the medical system to access a multileaf collimator (MLC) arc or a PET arc.

Another disclosed variation may include a removable crane installation system for servicing a medical system. The medical system comprises a couch, the couch including a couch patient platform and a couch mount, wherein the couch patient platform is attached to the couch mount via an attachment mechanism. Further, the removable crane installation system includes a removable crane, configured to attach to the attachment mechanism of the platform base.

In another variation, the medical system includes a gantry and a crane permanently coupled to the gantry, the crane having a first configuration for use and a second stowed configuration. For example, a medical system may include a gantry and a crane permanently coupled to the gantry, where the crane may have a first configuration for use and a second stowed configuration. The crane may include a tower and a jib, and where in the second stowed configuration the jib of the crane folds from a substantially perpendicular direction to the tower to a direction that forms at most forty-five-degree angle with the tower. The system may further include a cover configured to cover the gantry, components of the imaging system, and the crane. Alternatively, or additionally, the tower may be mounted at a first angle to a surface of the gantry, and where the first angle is an angle between the jib in the folded configuration and the tower, such that the jib is substantially parallel to a tangent direction to the surface of the gantry. The jib of the crane may be configured to rotate about an axis passing through the tower of the crane. In some variations, the medical system gantry may further comprise a drum rotatably coupled to a stationary frame of the gantry and a component coupled to the drum. In this variation, the jib may be coupled to the tower is by one of a hinge, a pivot, or a joint such that the jib is configured to rotate towards the drum and have an extension necessary to allow lifting the component.

The medical system may further have a drum rotation mechanism capable of rotating the drum, and the crane may further include a lock mechanism configured to interface with the drum rotation mechanism, such that the lock mechanism is engaged when the crane is in the second stowed configuration, and the drum rotation mechanism is configured to engage to rotate the drum when the lock mechanism is engaged and to stop a rotation of the drum when the lock mechanism is not engaged. The lock mechanism may include a switch having a button, and when the crane is in the second stowed configuration, an element of a crane is configured to depress the button resulting in the engagement of the drum rotation mechanism to rotate the drum.

Another disclosed variation may include a method for attaching a removable crane to a couch of a medical system. The method includes installing a couch coupling mechanism onto the couch, connecting a removable crane with the couch coupling mechanism, where connecting the couch coupling mechanism and the removable crane includes sliding an extension element of the removable crane into an opening of the couch coupling mechanism. Connecting the couch coupling mechanism and the extension element of the removable crane may further include positioning a tower of the removable crane at a first angle relative to a normal direction to a platform of the couch prior to the sliding of the extension element, and positioning the tower of the removable crane at a second angle relative to the normal direction to the platform, after the sliding of the extension element, resulting in a completion of an installation of the removable crane. In some variations, the difference between the first and the second angle may be at least five degrees.

### BRIEF DESCRIPTION OF THE DRAWINGS

FIG. 1 is a block diagram representation of one variation of a removable crane.

FIG. 2A is a schematic view of a variation of a removable crane attached to a couch.

FIG. 2B is a cross-sectional view of one variation of a removable crane attached to a couch and placed within a bore of a drum of a medical system.

FIGS. 3A-3C are various view of one variation of a removable crane.

FIG. 4A depicts one variation of a removable crane displayed while lifting a component from a drum of a medical system.

FIGS. 4B-4C are views of a variation of a couch and variations of coupling mechanisms for the couch.

FIG. 4D is a schematic view of one variation of a process of attaching a removable crane to a couch.

FIGS. 4E-4F are cross-sectional views of one variation of an extension element of a removable crane and a coupling mechanism of a couch.

FIG. 4G is an isometric view of one variation of an extension element of a removable crane.

FIG. 4H is a view of one variation of a base of a removable crane.

FIG. 4I is a view of one variation of a removable crane attached to a couch.

FIG. 4J is a view of one variation of a couch coupling mechanism.

FIG. 5 is one variation of a removable crane configured to attach to a couch at a location that is not at an end of the couch.

FIG. 6 is another variation of a removable crane configured to attach to a couch at a location that is not at an end of the couch.

FIG. 7 is one variation of a removable crane configured to move laterally relative to a surface of a couch, and also configured to rotate about an axis passing through a tower of the removable crane.

FIG. 8A is a view of one variation of a couch configured to be inserted into a bore of a medical system.

FIG. 8B is a view of one variation of a removable crane with a clamp element.

FIG. 8C is a view of one variation of a clamp element engaging with a component of a medical system.

FIG. 9 is a schematic view of one variation of a removable crane configured to lift a component of a medical system using multiple hooks.

FIGS. 10A-10B are flowchart representations of variations of methods for coupling a removable crane to a couch.

FIG. 11 is a block diagram representation of one variation of a crane mounted onto a support frame of a medical system.

FIG. 12A is a view of one variation of the crane mounted onto a support frame of a medical system.

FIG. 12B is a schematic view of one variation of a tower of a crane connected to a support frame of a medical system.

FIGS. 12C-12F are views of one variation of a crane mounted onto a support frame of a medical system in an operational and stowed configuration.

FIG. 13A is a view of one variation of a crane in a stowed configuration.

FIG. 13B is a view of one variation of a crane in an operational configuration.

FIGS. 13C-13E are views of a variation of a joint between a jib and a tower of a crane.

FIG. 13F is a cross-sectional view of one variation of a crane.

### DETAILED DESCRIPTION

Disclosed herein are variations of systems and methods comprising a crane for lifting components of a medical system. The medical system may include a positron emission tomography (PET) device and/or a computed tomography (CT) device, and/or a magnetic resonance imaging (MRI) device or any other medical device that includes a bore (e.g., a radiotherapy system). The medical system may include various components that might need to be lifted by a suitable crane. In some variations, the cranes disclosed herein are configured to be coupled (mounted) to a movable couch of a medical system and in other variations, the crane may be mounted on a supporting frame for supporting a drum of the medical system. When mounted to the movable couch, the crane may be configured to lift components of the medical system from within a bore of the medical system. For example, in some variations, the crane may include a jib configured to be placed within the bore of the drum for lifting components of the medical system (CMS). For example, the jib of the removable crane may be configured to be moved into the bore to access a multileaf collimator (MLC) arc or a PET arc. When the crane is mounted on the supporting frame, the crane may be configured to lift the CMS located on a side of the drum. Additionally, the crane mounted on the supporting frame may also be configured to lift at least some CMS located within the bore of the drum. The crane may be made from any suitable material (e.g., plastic, metal, glass, ceramics, combination thereof, and the like). For cases when an MRI system is being serviced, the crane may be made from non-magnetic materials.

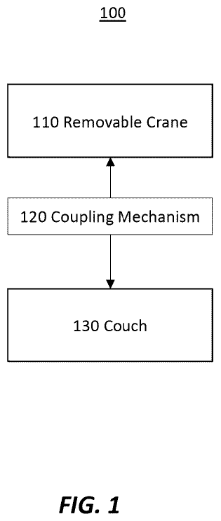

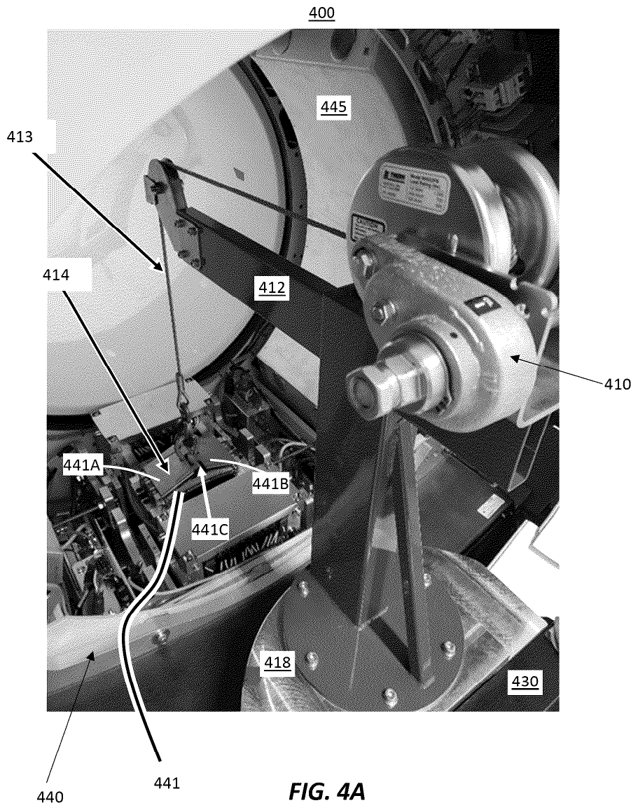

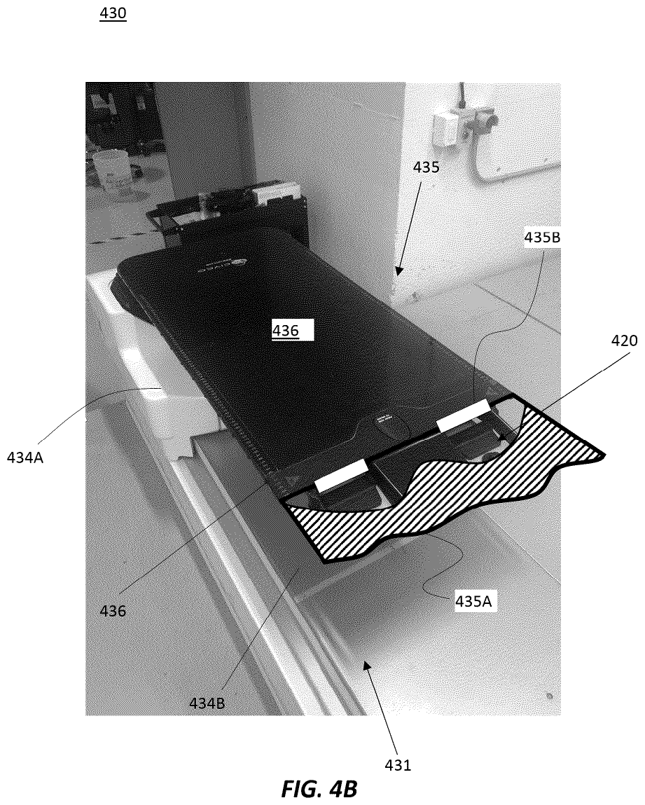

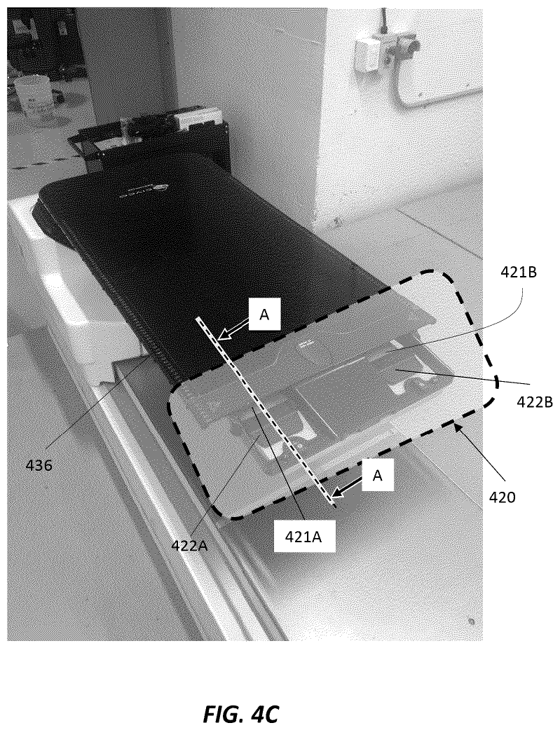

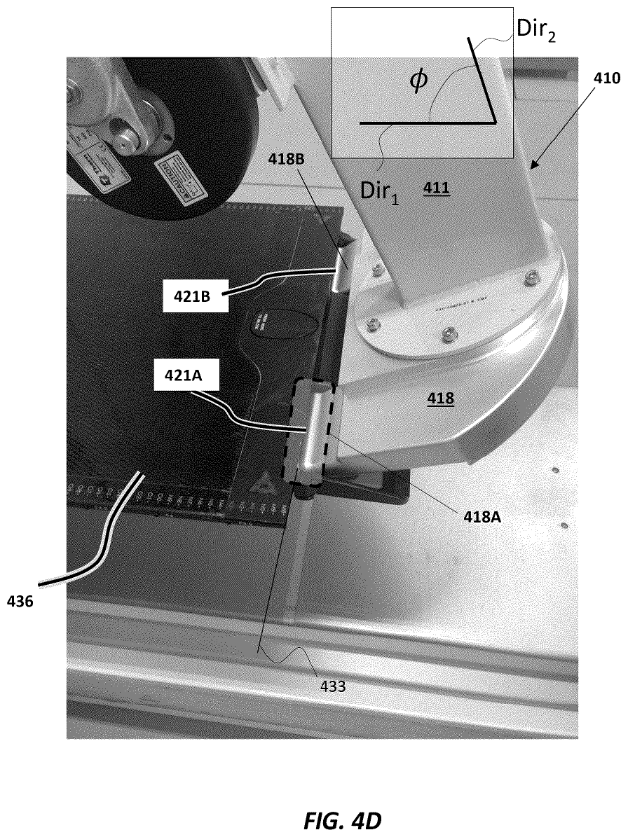

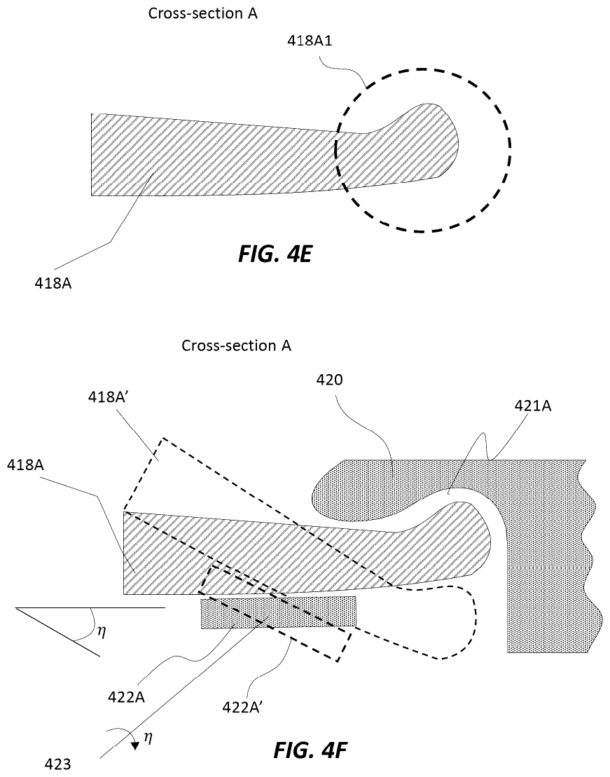

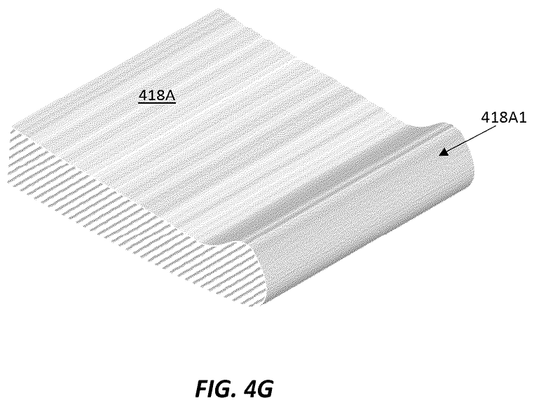

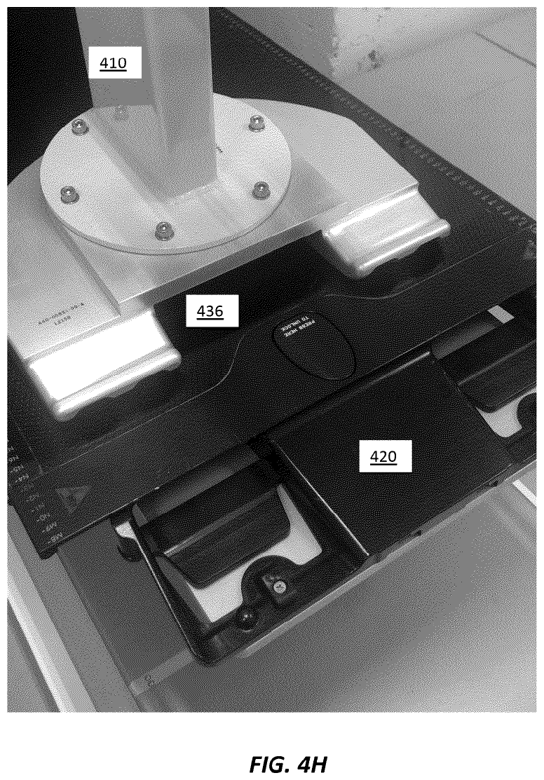

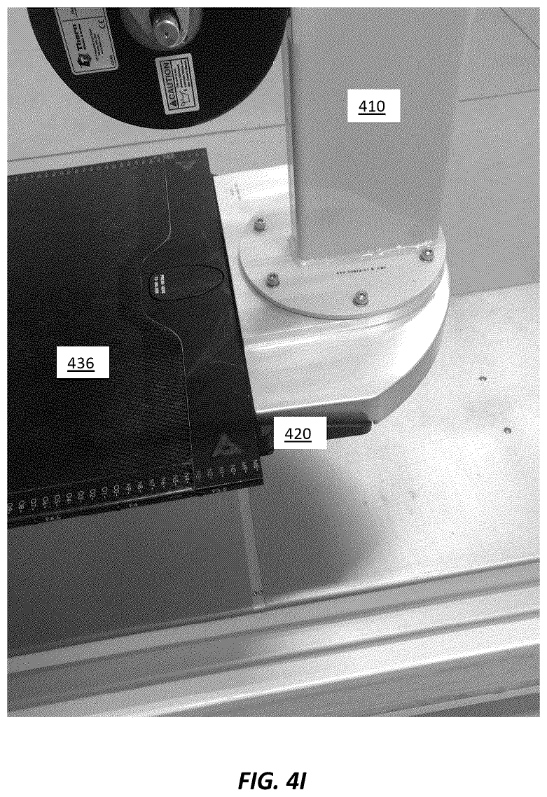

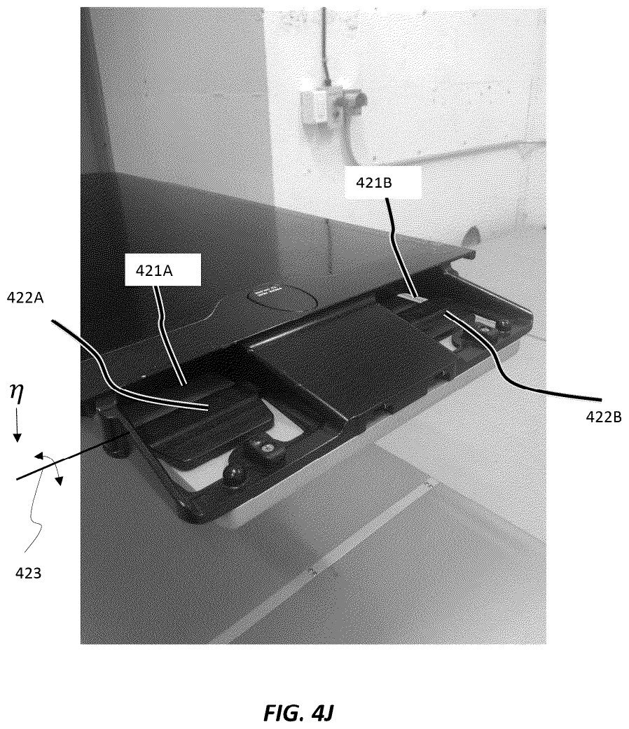

An example system 100 for maneuvering CMS (e.g., removing and/or installing) is shown in FIG. 1. The system 100 includes a removable crane 110 for servicing a medical system. The removable crane 110 may be configured to couple to a couch 130 of the medical system, wherein upon securing the removable crane 110 to the couch 130, a jib of the removable crane 110 is positioned to enter a bore of a drum of the medical system upon a movement of the couch 130 towards the drum. As depicted in FIG. 1, the removable crane 110 may be coupled to the couch 130 via a suitable coupling mechanism 120. Various implementations of the coupling mechanism 120 are further described below in connection with FIGS. 4C and 4D. It should be appreciated that the crane 110 can be compact and can be mounted to and dismounted from the couch 130 with ease (e.g., within a few minutes, a minute, or less than a minute). In some variations, when the couch 130 provides a sufficient storage, the removable crane 110 may be stored within the couch. The removable crane 110 may weigh a few tens of pounds (e.g., 20 pounds, 30 pounds, 40 pounds, 50 pounds and the like), and may lift as much as a few hundreds of pounds of weight, such as 100 pounds, 200 pounds, 300 pounds, 400 pounds, 500 pounds, and the like. In some cases, coupling mechanism 120 is configured such that mounting and dismounting the removable crane 110 to the couch may be performed by a single person.

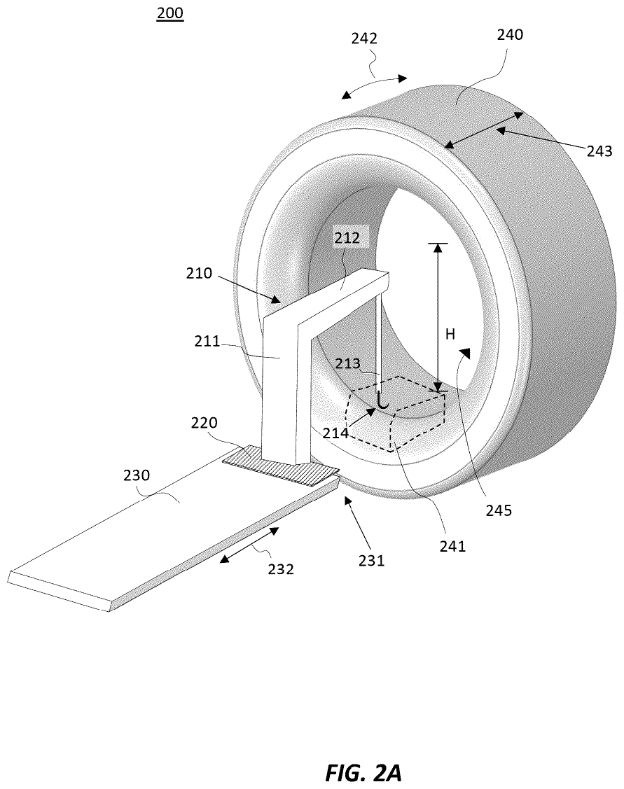

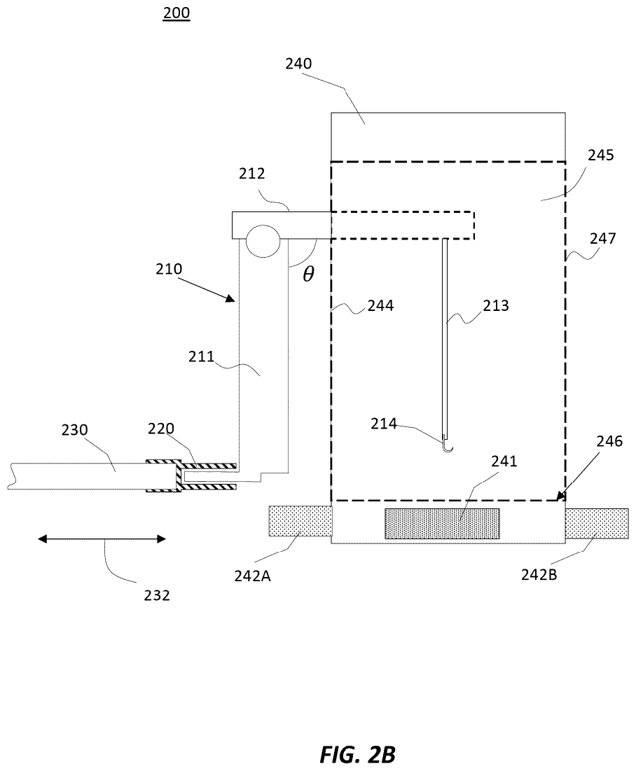

FIG. 2A shows an example system 200 which is a variation of the system 100. System 200 includes a removable crane 210 attached via a coupling mechanism 220 to a patient platform 230 of a movable couch. In one implementation, a movable couch may have a patient platform (e.g., platform 230) and a couch mount to which the platform is attached. In the example implementation, the couch mount is configured to support the patient platform may include one or more motion drive systems that are configured to move the patient platform into and out of a bore 245 of a drum 240. Optionally, the couch mount may have one or more motion drive systems that are configured to roll, pitch, and/or yaw the patient platform. Further, the patient platform is configured to support a patient, and may comprise a platform base that is attached to the couch mount and a platform extension that may be detachable from the platform base. FIG. 4B depicts an example of a couching having a patient platform that has two distinct parts or entities (i.e., the couch base and the patient platform are separable from each other). Alternatively, in some implementations, the patient platform may be a single elongated platform that cannot be separated into two distinct parts (i.e., an inseparable entity). FIG. 2A depicts an example of couch having a patient platform 230 that is an inseparable entity where the platform base and the platform extension are fixedly attached to each other (e.g., a unibody patient platform or otherwise fixedly connected). The removable crane 210 is configured to be positioned adjacent to the drum 240 of a medical system. The drum 240 may have a shape of an elongated cylinder having a longitudinal lumen or bore having a length as indicated by arrow 243. In the example implementation, as shown in FIG. 2A, the removable crane 210 is configured to be coupled to a distal end 231 of the patient platform 230 adjacent to the drum 240. The removable crane includes a tower 211, a jib 212 connected to the tower, and in some cases, substantially perpendicular to the tower, a hoist line 213 connected to the jib 212, and a hook 214 attached to the hoist line 213. In some variations, the jib may be an elongate element that is connected at an angle to the tower, where a proximal end of the elongate element is attached to the tower and the hoist line is attached to the distal end of the elongate element. The hook 214 may be configured to engage with an opening or loop of a CMS such as a component 241 to lift it from the drum 240, as shown in FIG. 2A. In some variations, the drum 240 may be configured to rotate as indicated by arrow 242 (i.e., around the longitudinal axis of the bore 245), thus allowing for the component 241 to be placed underneath the hook 214. In some cases, component 241 includes a suitable coupling region with, for example, an opening to which the hook 214 may be attached. Alternatively, if such an opening is not available, a harness may be placed around component 241 to which the hook 214 may be attached. It should be appreciated, that hook 214 can be substituted with any other suitable coupling device for coupling to components of the medical system. For example, instead of hook 214 an electromagnet may be used, or a coupling device that can couple to a coupling component of the medical system via screws, clamps, or any other coupling means. In some cases, a plurality of coupling devices (e.g., a hook and a magnet, a clamp, and the like) can be used together to couple to a component of the medical system. In some cases, a first coupling device (e.g., a hook) may be configured to couple to the first coupling component of the medical device and a second coupling device (e.g., another hook, or a magnet, or a clamp) may be configured to couple to the second coupling component of the medical device.

As shown by an arrow 232, the couch 230 is configured to move into and out of the bore 245 (i.e., along the longitudinal axis of the bore). The crane 210 may be first mounted to the couch 230 when the couch is moved away from the drum 240. Subsequently, the couch 230 is moved towards the drum 240 such that the jib 212 of the crane 210 is placed within the drum with hoist line 213 positioned substantially perpendicular to a surface of the component that requires lifting when hook 214 is coupled to the component. In some variations, a height of tower 211 may be adjusted such that the jib 212 fits within the drum 240. Alternatively, the height of tower 211 may be selected such that the jib 212 is located in an upper part of the drum 240 and a height H, as shown in FIG. 2A, such that any bulky component of the medical system is configured to fit within the space defined by height H, when lifted from within the bore 245 of the drum 240.

FIG. 2B shows a cross-sectional view of system 200 including the couch platform 230, the coupling mechanism 220, the removable crane 210, the drum 240, the bore 245, and the component 241. In some variations, the coupling mechanism may be integrally formed with the platform. As shown, the couch platform 230 is configured to move into and out of the bore 245, such that the jib 212 is configured to move into or completely out of the bore 245. In various implementations, the jib 212 may be configured to reach the distal-most end (e.g., point 246) of the bore of the drum for lifting the components located near a distal rim 247 of the drum 245. In some cases, the jib 212 may be sufficiently long to extend past the distal rim 247 of the drum 240 (when the couch 230 is moved sufficiently close or even within the drum 245) to lift a component 242B located on an outside of the distal rim 247 of the drum 240. Alternatively, the crane 210 may be configured to be able to lift a component 242A located outside a proximal rim 244 of the drum 240. For example, the couch 230 may be moved sufficiently far away from the drum 240 such that the hook 214 suspended from the hoist 213 is located directly above the component 242A.

It should be appreciated that the jib 212 of the crane 210 may have an adjustable length (e.g., the jib 212 may be shortened or extended as needed. Additionally, in some cases, the orientation of the jib 212 relative to tower 211 may be adjusted. For example, the jib 212 may be at an angle θ to the tower 211 that may be 60°, 70°, 80°, 90°, 100°, 110°, 120°, and the like. Further, the height of the tower 211 may be adjustable, as described above.

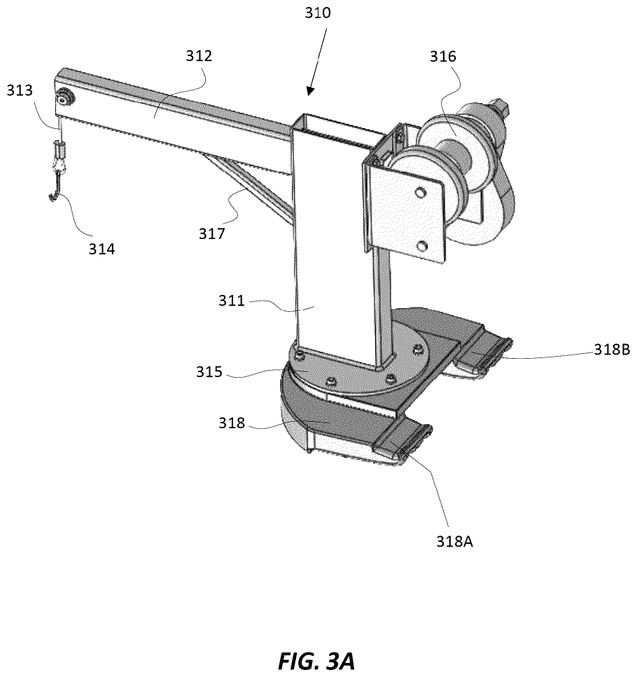

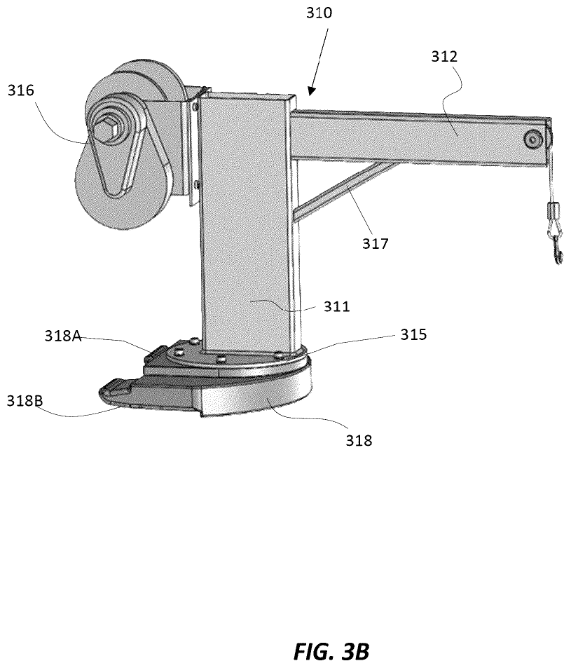

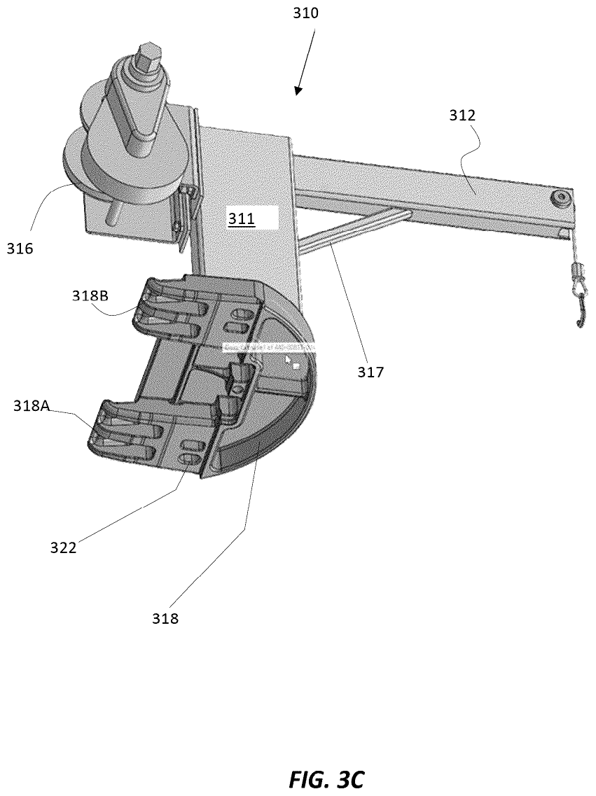

FIGS. 3A-3C show various perspective views of an example removable crane 310. Removable crane 310 may be the same or similar in form or in function to the removable crane 210. FIG. 3A shows a top-side view of the crane 310. The crane includes a jib 312 and a tower 311, which may be made from a suitable high-strength material such as steel, aluminum, duralumin, and the like. In one variation, the tower 311 may have a shape of a rectangular or circular cylindrical shell (the rectangular cylindrical shell forming the tower 311 is shown in FIG. 3A). Such shape may allow for a high structural strength, while maintaining a relatively light weight. Other well-known structural shapes for the tower 311 and the jib 312 may be used such as I-beams, or similar structures (e.g., IPN beams) having flanges and web elements, or columns of various cross-sections (e.g., rectangular cross-section). In some variations, the crane 310 may include a winch 316 that may be geared (e.g., the winch 316 may be a worm-gear winch). The torque applied on a crank of the winch 316 depends on the gear ratio or number of teeth on the gears that are used by winch 316. In an example implementation, the winch 316 may have a first gear on an axle attached to the crank (herein, referred to the input axle), and a second gear attached to an axle of the wheel (herein, such axle is referred to as an output axle) for adjusting the tension on the hoist line to pull it in (e.g., by increasing the tension) or to deploy it (e.g., by releasing the tension). A higher gear ratio (e.g., the number of revolutions a crank makes for a single revolution of the second gear) may facilitate smooth and/or quiet operation of the winch 316. In an example implementation, a gear ratio may be in a range of 4-to-1 to 22-to-1. For example, the gear ration may be 9-to-1, 10-to-1, 11-to-1, 12-to-1, 13-to-1, 14-to-1, 15-to-1, 16-to-1, 17-to-1, and the like. In some implementations, the winch 316 may be powered with a crank, a socket wrench or a cordless drill with a socket. As shown in the example depicted in FIG. 3A, the jib 312 is fixed to (e.g., unmovable relative to) tower 311, and is further supported by a supporting strut 317. The strut 317 provides a counter-torque to the torque due to the weight of a component of the medical system when it is suspended from a hook 314 attached to a hoist line 313.

The crane 310 may further include a base plate 315 which can be coupled to the base 318 in any suitable manner or using any suitable attachment mechanism. For example, the base plate 315 may be welded to the base 318 or clamped, or bolted, as shown in FIG. 3A. In some cases, the base plate 315, the base 318, and the tower 311 may be fabricated from a single sheet of metal (e.g., steel, aluminum, duralumin, and the like), such that these elements do not need to be joined together. In one variation, the base plate 315 and the base 318 are designed to be the heaviest part of the crane 310. For example, the base plate 315 and the base 318 may weight a few tens of pounds (e.g., 30 lbs, 40 lbs, 50 lbs, and the like). The base 318 may include extension elements 318A and 318B configured to fit into an opening (or openings) of a coupling mechanism (e.g., the couch coupling mechanism 220), as further described below in connection to FIG. 3B. FIG. 3B shows a side view of the crane 310, and FIG. 3C shows a bottom-side view of the crane 310 with elements 311-318B labeled as in FIG. 3A. In an example implementation, as shown in FIG. 3C, the bottom side of the crane 310 includes optional cavities 322. The presence of the optional cavities 322 may reduce a weight of the base 318, while maintaining the structural strength of the base 318.

FIG. 4A shows an overall perspective view of medical system 400 and a removable crane 410 attached to a couch 430 of the medical system. A base 418 of the removable crane 410 is moved adjacent to a rim of a drum 440 of the medical system 400 by advancing the couch 430, such that a jib 412 of the removable crane 410 is located inside a bore 445 of the drum 440. A hoist line 413 is positioned substantially perpendicular and downwards from the jib 412. In one variation, as shown in FIG. 4A, the removable crane 410 may include a clamp element 414 that is configured to couple to a component 441 located inside the drum 440 of the medical system 400. The clamp element 414 is configured to grip component 441 from both sides of the component 414, as shown in FIG. 4A, as opposed to a hook element, configured to hook a particular coupling element (e.g., a wire loop) of the component 414. In an example implementation, the clamp element 414 includes two grasping structures 441A and 441B connected at a hinge 441C, and the hinge 441C can be locked once a component is retained between the two grasping structures 441A and 441B.

FIG. 4B shows an example couch 430 that has a couch mount 434A and a patient platform 435 attached to the couch mount 434A. The couch mount 434A may have a drive motor (e.g., an electromotor) that is configured to move the couch mount 434A along a guide element 434B. Optionally, the couch mount may comprise a plurality of drive motors configured to position the patient platform in a desired orientation, as described above. The couch 430 may be the same or similar in form or in function to the couch 230, as shown in FIGS. 2A and 2B. The patient platform 435 has platform base 436 that is fixedly coupled to the couch mount 434A and a platform extension 435A that is configured to attach to the distal end 431 of the platform base 436 via a couch coupling mechanism 420. The attachment between the platform extension and the platform based may be releasable such that the platform extension may be removed when it is not in use. The platform extension 435A can be mounted onto the platform base 436 by attaching a proximal end of the platform extension portion 435A to the distal portion of the platform base 436 and connecting a coupling mechanism 435B associated with the platform extension portion 435A to the couch coupling mechanism 420 of the platform base 436. FIG. 4C shows the platform base 436 with the platform extension 435A removed. The platform base 436 includes the couch coupling mechanism 420 located at its distal end. The coupling mechanism 420 includes sockets (herein, also referred to as openings) 421A and 421B, and guiding elements 422A and 422B.

In one variation, the removable crane 410 may be configured to be coupled to the coupling mechanism 420 of the platform base 436. The same motion mechanisms (e.g., motors) used to control the motion of the patient platform 435 may be used to advance the removable crane 410 into the bore to access the CMS, e.g., multi-leaf collimator (MLC) and/or PET arcs.

FIG. 4D shows the removable crane 410 being coupled to coupling mechanism 420 by inserting extension elements 418A and 418B of the base 418 into sockets 421A and 421B. To couple the removable crane 410 to coupling mechanism 420, the crane 410 is tilted by an angle, as shown in FIG. 4D. Angle ϕ is the angle between a direction Dir1 that is parallel to a surface of the couch 430 (and perpendicular to an edge 433 of the couch base 436, as shown in FIG. 4D), and a direction Dir2 that extends along an axial direction of a tower 411 of the removable crane 410. In one variation, angle ϕ may depend on a particular form and shape of extension elements 418A and 418B, and may be, for example, in a range of a few degrees to about 60 degrees. In some variations, angle ϕ may be larger than five degrees.

It should be appreciated that sockets 421A and 421B may be one example of a coupling. In an alternative variation, the couch coupling mechanism may include one or more grooves, and the removable crane may include an extension element configured to slidably engage with the groove of the couch coupling mechanism. Further, the extension element may be secured in a grove by means of a pin.

FIG. 4E shows a cross-sectional view of the extension element 418A. The cross-section is taken along line A, as shown in FIG. 4C. In the variation shown, the extension element 418A includes a bent portion 418A1 configured to be inserted into the socket 421A of the coupling mechanism 420, as shown in FIG. 4F. FIG. 4F shows the cross-sectional view (along line A, as shown in FIG. 4C) of the coupling mechanism 420. As shown in FIG. 4F, the extension element 418A′ in a tilted configuration while being inserted into the socket 421A and the extension element 418A in a horizontal position after being coupled to the socket 421A. Further, FIG. 4F shows the guiding element 422A (as previously shown in FIG. 4C, and herein also referred to as a flap 422A) supporting the extension element 418A. The flap 422A may be configured to rotate about axis 423 during the insertion of the extension element 418A and is shown in a rotated configuration as a flap 422A′. The angle by which flap 422A is rotated into configuration 422A′ is η and is the same as angle ϕ, as shown in FIG. 4D. When the removable crane 410 is tilted by an angle ϕ and the extension elements 418A and 418B are inserted into the sockets 421A and 421B, flaps 422A and 422B are configured to rotate by the angle ϕ, and when the removable crane 410 is placed upright, thereby securing the extension elements 418A and 418B in the sockets 421A and 421B, flaps 422A and 422B are configured to rotate back to a horizontal configuration (η=0 degrees). It should be noted that difference between angle of the tilted position for the removable crane 410 and the upright position may be more than five degrees.

FIG. 4G shows a perspective view of extension element 418A with the bent portion 418A1. The extension element 418B may be configured in the same way as the extension element 418A. FIG. 4H shows a view of the removable crane 410 removed from the coupling mechanism 420 and placed onto the couch base 436 to further illustrate the scale of the removable crane 410 relative to the coupling mechanism 420 and the couch base 436, FIG. 4I shows the removable crane 410 coupled to the coupling mechanism 420 of the couch base 436, and FIG. 4J shows further details of the coupling mechanism 420. In an example implementation, the coupling mechanism 420 may include guiding elements (herein also referred to as flaps) 422A and 422B, as previously described in relation to FIGS. 4C and 4F. As previously described, these elements may be configured to guide extension elements 418A and 418B into sockets 421A and 421B.

It should be appreciated, that coupling mechanism 420 is one possible mechanism for coupling the removable crane 410 to a platform base 436, and various other coupling mechanisms may be used alone or in combination with coupling mechanism 420. For example, in some implementations, when a medical couch is separable into a platform base and a platform extension, a removable crane may be clamped directly to the platform base via suitable clamps. In some variations, the couch coupling mechanism may comprise one or more extensions or protrusions, and the coupling mechanism of the removable crane may comprise one or more corresponding recesses or grooves that mate with the couch coupling mechanism. The coupling mechanism of the removable crane may be the same as, or similar to, the coupling mechanism of the platform extension. The couch coupling mechanism and/or coupling mechanism of the crane may optionally comprise a lock that secures the engagement between the coupling mechanisms of the couch and the crane. Optionally, there may be one or more alignment or guiding structures, such as rails, grooves or protrusions to help guide a user to align the coupling mechanisms to each other.

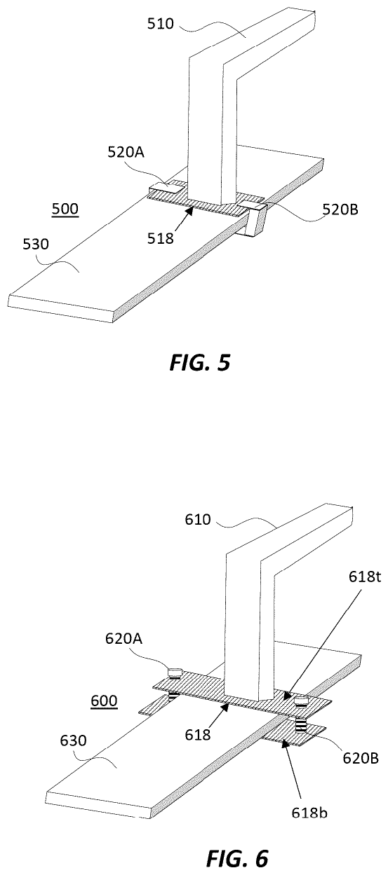

In variations of a medical system comprising a unibody patient platform, a couch coupling mechanism may be included at the distal-most end of the patient platform. Alternatively, or additionally, a coupling mechanism of the removable crane may comprise one or more clamps that are configured to releasably attach to the patient platform. For instance, FIG. 5 shows a couch 500, and a removable crane 510. The removable crane 510 may be similar to the removable cranes 210-410 but may have a different base. For example, the removable crane 510 may include a substantially flat base 518 which may be clamped via coupling clamps 520A and 520B to the patient platform 530 of the couch 500.

FIG. 6 shows another variation of a couch 600 (similar to the variation shown in FIG. 5). A removable crane 610 coupled to a patient platform 630 of the couch 600 via bolts 620A and 620B and the flat top and bottom respective base elements 618t and 618b. It should be noted that any one of the coupling mechanisms, as shown in FIGS. 5 and 6, may not necessarily be installed at the distal end of the couch but may be installed at any part of the couch close to the distal end of the couch, in order for the jib of the removable crane to reach the inside of the drum of the medical system. For example, the couch coupling mechanism may be configured to be attached at a location along the length of the patient platform located between a substantially middle position of the couch and the distal end of the couch.

It should be further appreciated that other coupling mechanisms may be used for coupling a removable crane to a couch, e.g., a patient platform of a couch. For example, a frame (e.g., an A-frame) may be placed on either side of the couch, and a beam may be placed across the bore between the two frames. The removable crane then may be attached to the beam and may be moved along the beam via cables and a trolley. It should be further appreciated that that a coupling mechanism may be separate from the couch and the removable crane and may be configured to attach to various couch platforms. Upon attachment of the coupling mechanism, the removable crane is then configured to couple the coupling mechanism.

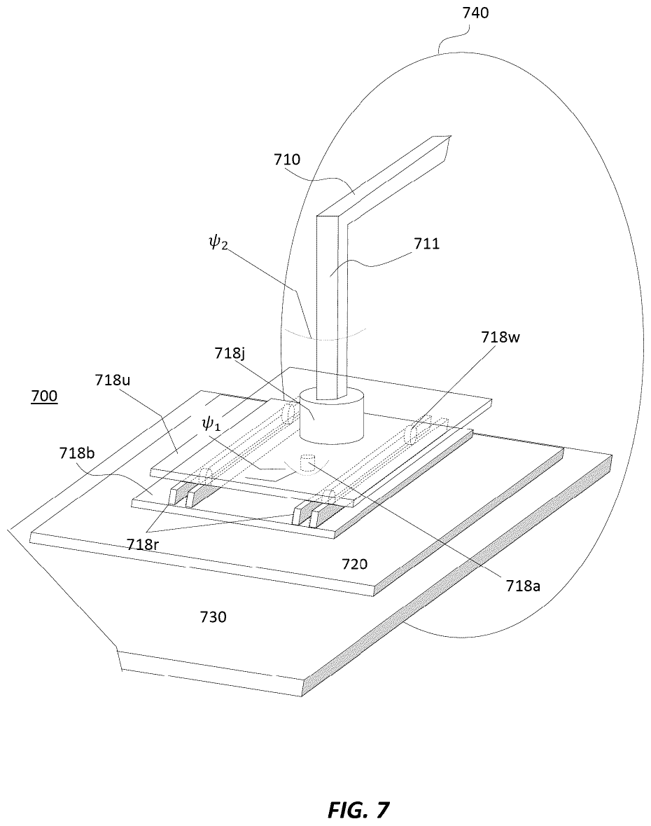

In any of the variations described herein, a removable crane may be capable of lateral and rotational motion relative to a couch patient platform. For instance, FIG. 7 depicts a removable crane 710 capable of lateral and rotational motion relative to couch 700 (i.e., platform, platform base and/or platform extension). The removable crane 710 may be attached to a coupling mechanism 720 of the couch 700 (e.g., located on platform 730), and the base 718 of the removable crane may include a bottom part 718b, an upper part 718u, a set of rails 718r and a set of wheels 718w, with the wheels 718w configured to move along the rails 718r. In one optional variation, bottom part 718b is configured to rotate around axis 718a by a first angle ψ₁ and the tower 711 of the removable crane 710 optionally includes a joint element 718j configured to rotate about the upper part 718u by an angle ψ₂, thereby providing a rotation for the tower element 711. Thus, angle ψ₁ determines a direction of a lateral movement relative to the patient platform 730, and angle ψ₂ rotates the crane relative to a drum 740.

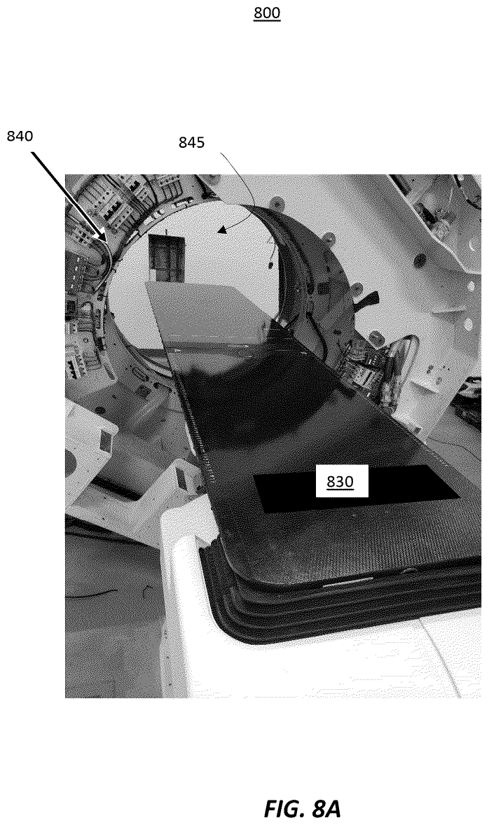

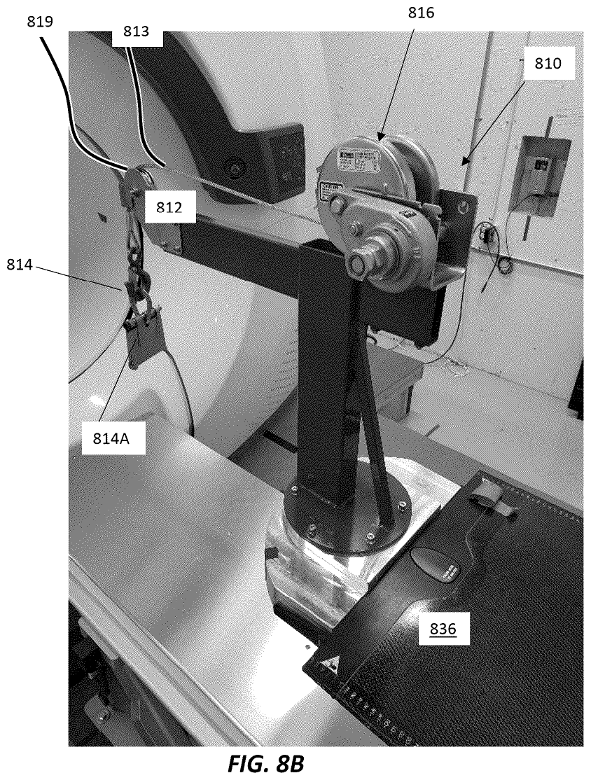

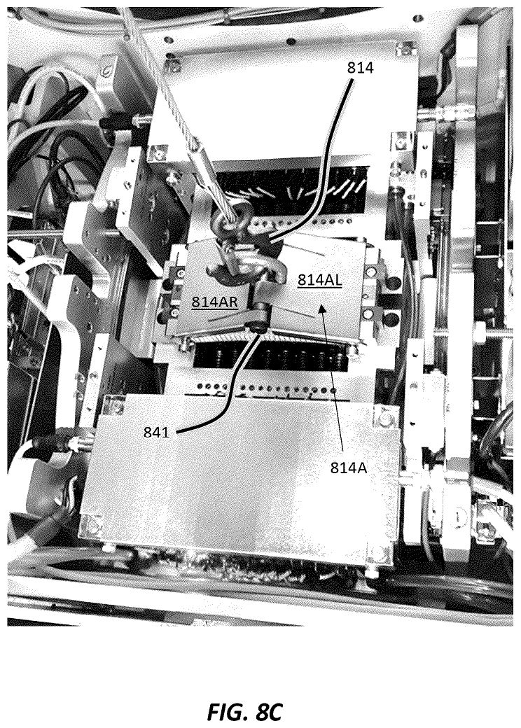

FIGS. 8A-8B show further details of a system 800 of a medical system. For example, FIG. 8A shows that that a couch 830 is configured to enter a bore 845 of a drum 840 of system 800. In one variation, couch 830 may have a couch mount (comprising one or more drive motors) and a patient platform, which may optionally comprise a platform base that is attached to the couch mount and a platform extension that is releasably coupled to the platform base, or may be unibody platform that has a couch coupling mechanism at its distal end. FIG. 8B shows that a removable crane 810 is attached to the platform base 836 using any of the couch coupling mechanisms described herein, and may include a hook 814 and a lifting clamp 814A suspended from the hook 814 (e.g., hook 814 may be the same as hook 314, as shown in FIG. 3A). In one variation, the clamp 814A may be configured to clamp around a loop of a wire connected to a component of the medical system located inside the drum 840. Alternatively, as shown in FIG. 8C, a left side 814AL and a right side 814AR of the clamp 814A are configured to grasp respective sides of component 841 of the medical system. As shown in FIG. 8B, hook 814 is attached to a hoist line 813 configured to pass through a pulley 819. The hoist line 813 is coupled to a winch 816 which is configured to pull (or release) the hoist line, thereby moving the hook 814 and the attached clamp 814A up towards a jib 812 (or down away from the jib 812).

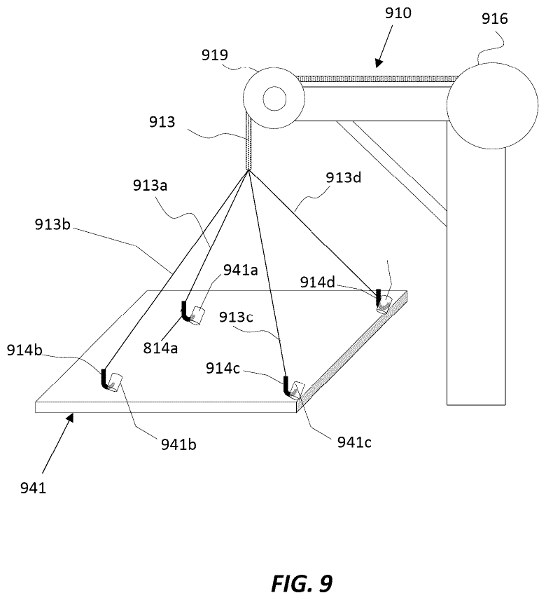

FIG. 9 depicts another variation of a system 900 including a component 941 (e.g., a multileaf collimator schematically indicated as a cubical block in FIG. 9) having several hook-receiving elements 941a-941d configured to be coupled to respective hooks 914a-914d of a removable crane 910. In the variation depicted in FIG. 9, hooks 914a-914d may be attached to respective hoist line segments 913a-913d which, for example, are attached to a master hoist line 913 (the master hoist line 913 may be similar or the same in form or in function to the hoist line 813, as shown in FIG. 8B). In some variations, receiving elements 941a-941d are positioned such that component 941 is in a stable orientation when it is lifted up by the removable crane 940 (e.g., the component 941 stays in the same orientation as it is being lifted up by the removable crane 940).

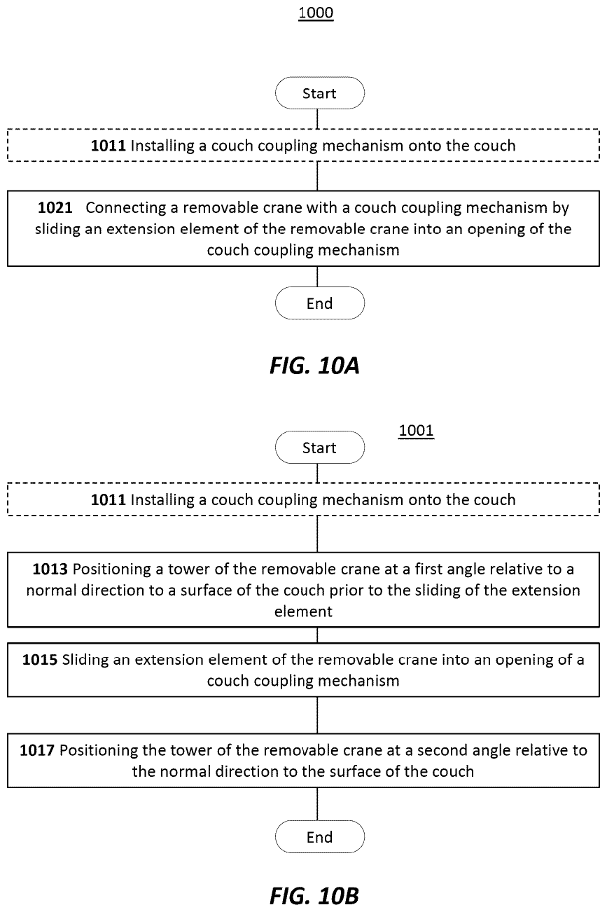

FIG. 10A is an example of a method 1000 of mounting a removable crane onto a couch for lifting a component of a medical system. The method may optionally include installing 1011 a couch coupling mechanism onto the couch. In one variation, the couch coupling mechanism may be separate from the removable crane and may be configured to be connected first to the couch, e.g., couch patient platform. In one variation, the couch coupling mechanism may be similar or the same as coupling mechanism 420, as shown in FIG. 4C. The method 1011 may further include connecting 1021 the removable crane with the couch coupling mechanism by sliding an extension element (or several extension elements) of the removable crane into an opening (or several openings such as sockets 421A and 421B, as shown in FIG. 4C).

FIG. 10B shows another example of a method 1001 of mounting a removable crane onto a couch for lifting a component of a medical system. Step 1011 of method 1001 may be the same as step 1011 of method 1000 and may be optional. The method 1001 includes positioning 1013 a tower of the removable crane (e.g., tower 411, as shown in FIG. 4D) at a first angle (e.g., angle ¢) relative to a normal direction to a surface of the couch (e.g., patient platform) prior to the sliding of the extension element (or several extension elements such as 418A and 418B, as shown in FIG. 4D). The method 1001 may include sliding 1015 an extension element of the removable crane into an opening (e.g., a socket 421A) of the couch coupling mechanism. The method 1001 may include positioning 1017 the tower of the removable crane at a second angle relative to the normal direction to the surface of the couch or patient platform (e.g., placing the tower 411 upright and perpendicular to the surface of the couch), which may lock the removable crane to the couch.

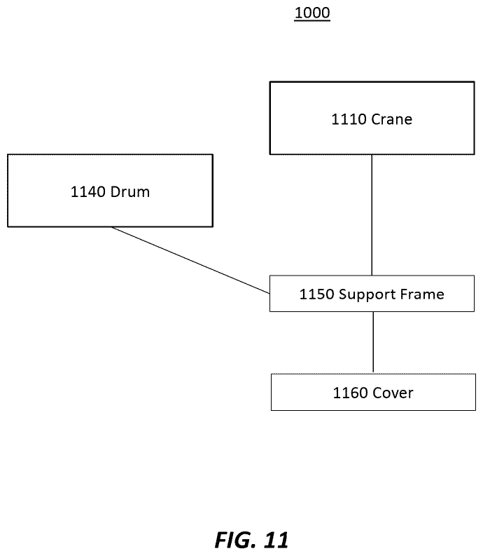

While FIGS. 1-10 disclose removable cranes coupled to a couch, other variations disclosed herein relate to a crane mounted to a support frame of the medical system. FIG. 11 shows schematically a system 1100 having a crane 1110 connected to a support frame 1150, which in turn supports a drum 1140. In some variations, the support frame may be a stationary frame while the drum may be rotatably coupled to the stationary frame. The crane 1110 is configured to lift various components of the medical system positioned within the drum 1140 (or adjacent to outside surfaces of the drum 1140) as further shown and explained in relation to FIG. 11. In some variations of the system 1100, the crane 1110 is configured to be part of the medical system, and thus, typically is not a removable component. Instead, the crane 1110 is configured to be stowed away during the operation of the medical system underneath a cover 1160 that encloses the support frame 1150 and the drum 1140. Thus, in the stowed configuration, the crane 1110 is configured to fit underneath the cover 1160. In some variations, the drum may be permitted to rotate only when the crane is in the stowed configuration, and/or may be prevented from rotating (e.g., locked) when the crane is in an operational configuration. For example, there may be one or more sensors that detect whether the crane is in the stowed configuration and if so, the drum may then be unlocked and able to rotate. Alternatively, or additionally, there may be one or more sensors that detect whether the cover is removed and if so, the drum may then be locked and prevented from rotating. In some variations, the system may comprise a mechanical interlock mechanism between the crane and the drum, which may include a mechanical obstruction to drum rotation when the crane is in an operational configuration and may optionally include a release mechanism that permits the drum to rotate when the crane is in a stowed configuration. During the maintenance of system 1100, the cover 1160 may be removed, and the crane 1110 may be released from the stowed configuration into an operational configuration. In some variations, the crane 1110 may be able to support several hundred pounds (e.g., 100 lbs, 200 lbs, 300 lbs, 400 lbs, and the like). The crane 1110 may weigh a few tens of pounds (e.g., 40-90 lbs), and having the crane 1110 permanently installed on the support frame 1150 alleviates efforts of installing a crane at the start of a service/repair session and removing the crane at the end of the service session.

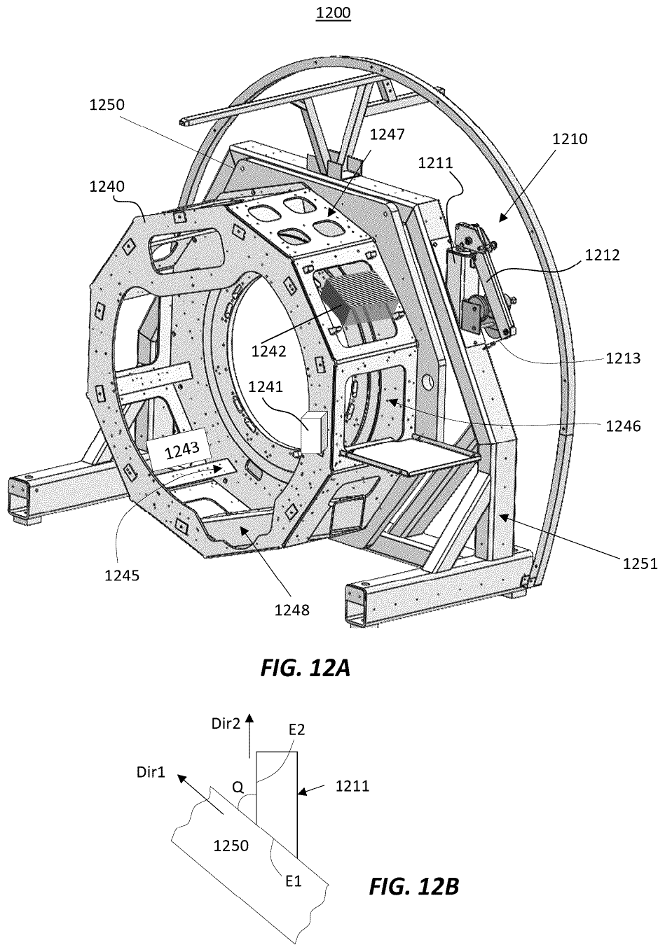

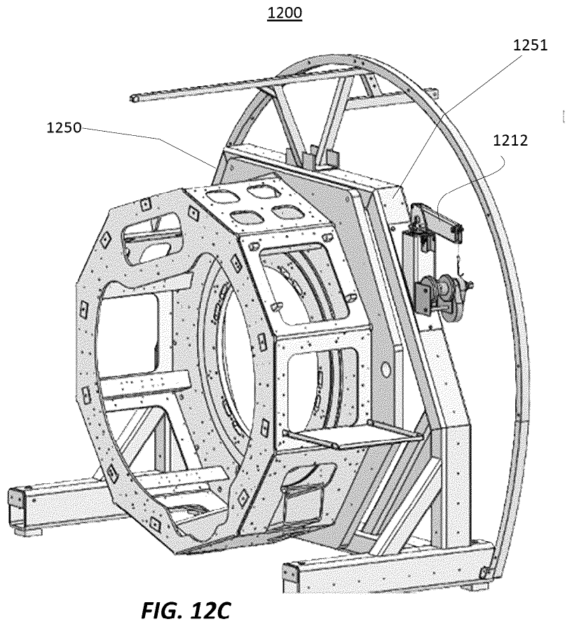

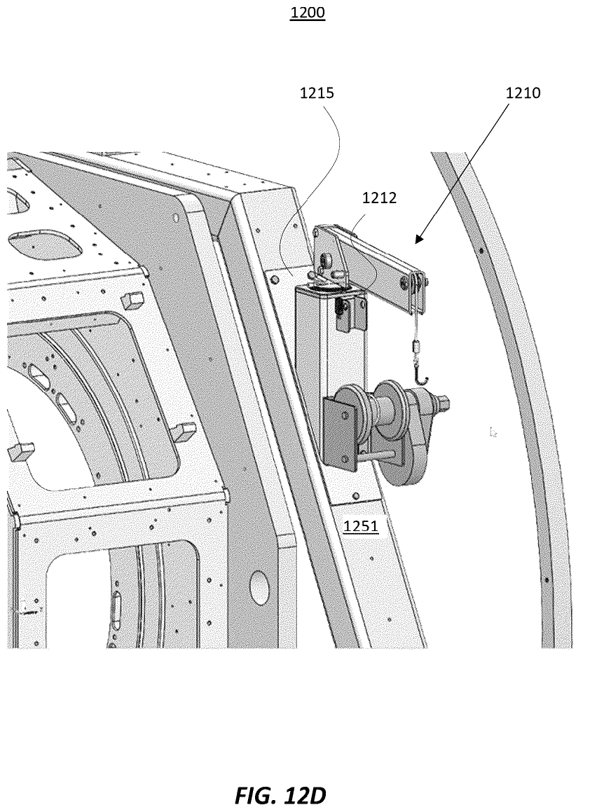

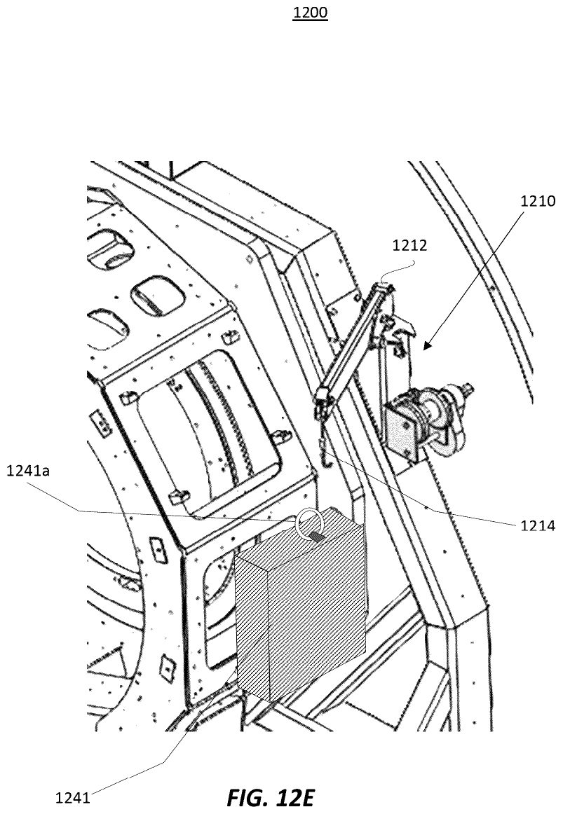

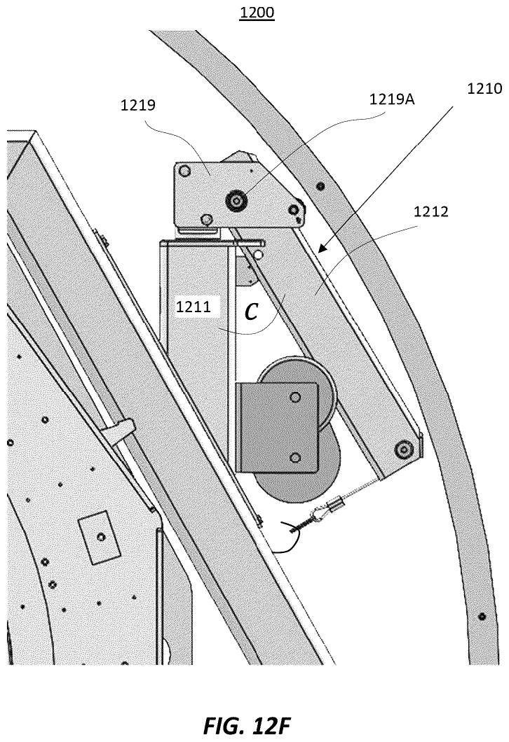

FIGS. 12A-12F show different views of a frame-mounted crane 1210 in a stowed configuration (e.g., FIG. 12A) and operational configuration (e.g., FIGS. 12C, 12E). The crane 1210 is mounted on a non-rotating support frame 1250 of the medical system 1200. FIG. 12A shows the crane 1210 in a stowed configuration and mounted on a side 1251 of the support frame 1250. In the stowed configuration a jib 1212 of the crane 1210 is moved towards a tower 1211 of the crane 1210 such that the jib 1212 is at an angle C to the tower 1211, as more clearly shown in FIG. 12F.

FIG. 12A further depicts the drum 1240 rotatably attached to the stationary support frame 1250. The drum 1240 includes components 1241 and 1242 that may be lifted by the crane 1210. In an example implementation, components placed on an outside part 1247 of the drum 1240 may be lifted (e.g., such as the component 1242) and components placed on an outer rim 1248 of the drum 1240 (e.g., such as the component 1241). In some cases, some other components (e.g., components located in a bore 1245 of the drum 1240) may be lifted by the crane 1210, for example, via an access opening of the drum 1240 (e.g., an access opening 1246, as shown in FIG. 12A). The hoist line 1213 may be lowered through the access opening to lift a component located on substantially opposite of the opening 1246 and inside the drum 1240 (e.g., a component 1243, as shown in FIG. 12A).

As shown in FIG. 12A, the tower 1211 is mounted on a surface 1251 of the support frame 1250. FIG. 12B shows a side view of the tower 1211 and the side of support frame 1250. As shown, the tower 1211 is mounted at an angle Q to the support frame 1250 (e.g., the angle Q is formed from a direction Dir1 along an edge E1 of the support frame 1250 and a direction Dir2 along an edge E2 of the tower 1211). In one variation, the angle Q may be 45 degrees or be in a range of 10-90 degrees depending on a particular shape of frame 1250. In some variations, the tower 1211 may be configured to stand upright (e.g., opposite to a direction of gravity).

FIG. 12C shows the crane 1210 in an operating configuration. In this configuration, the jib 1212 of the crane 1210 is rotated to be substantially perpendicular to the tower 1211. Further, as shown, in FIG. 12D, the crane 1210 includes a base plate 1215 having orientation parallel to the surface 1251. The base plate 1215 is solidly connected to the tower 1211 (e.g., the base plate 1215 is welded to tower 1211, or the tower 1211 and the base plate 1215 may be formed from the same sheet of metal), and the base plate 1215 is attached using bolts to the surface 1251 (or attached via any other suitable high-strength connection, such as welding, clamping, and the like).

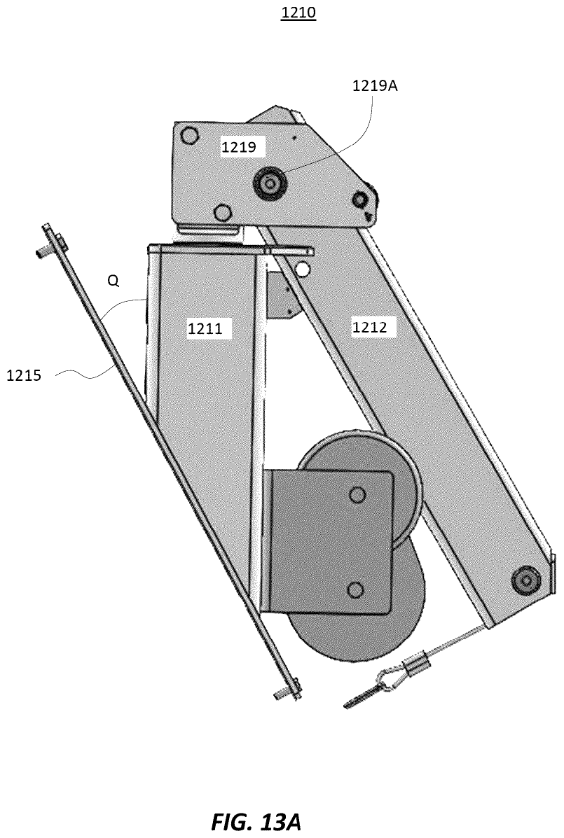

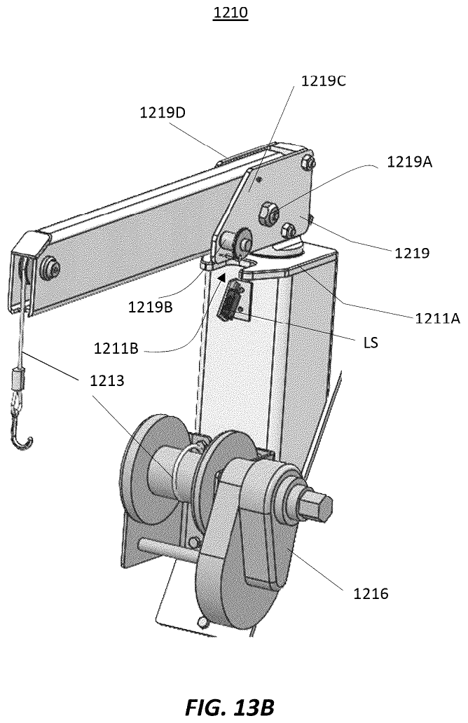

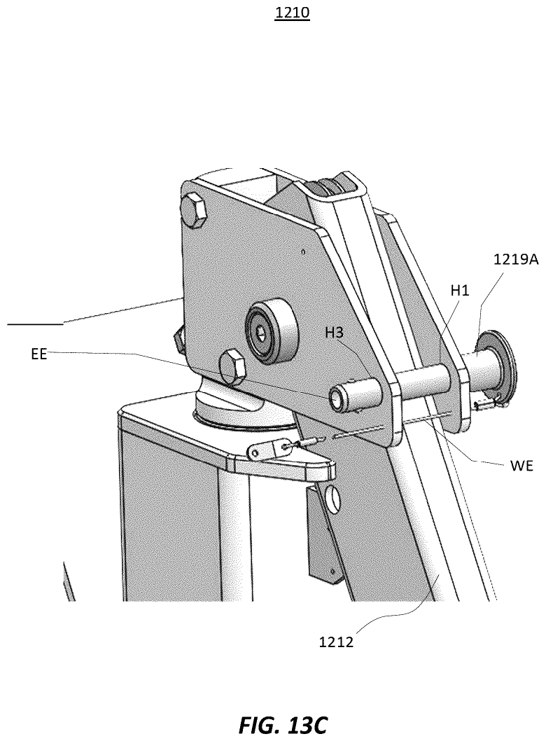

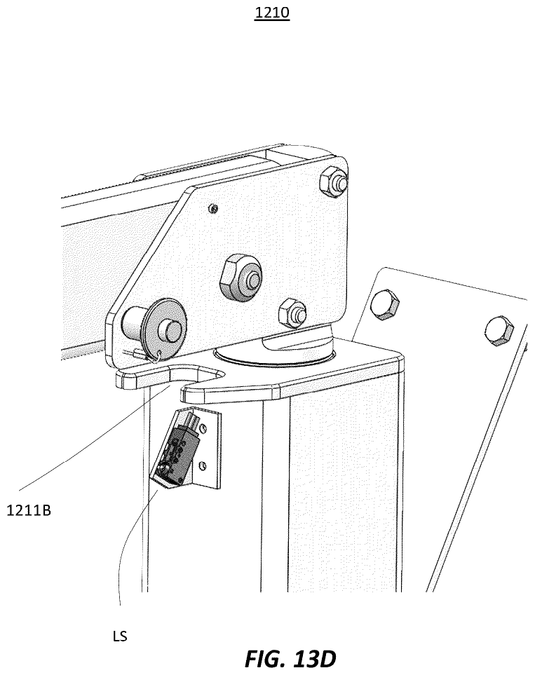

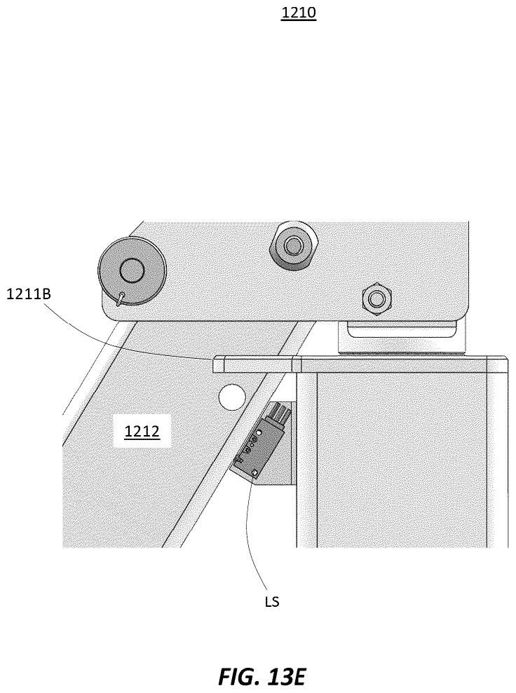

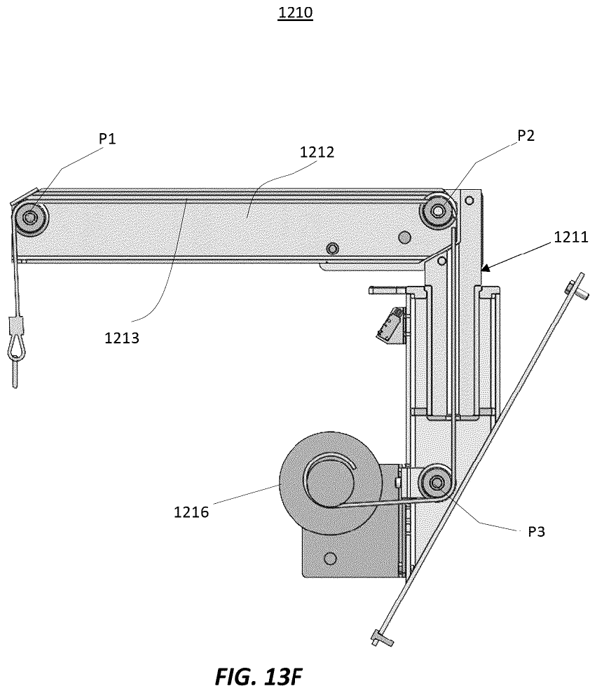

In order for the crane 1210 to lift various components off the drum 1240, the jib 1212 of the crane 1210 is configured to rotate about tower 1211. In some variations, the jib may be pivotably mounted to the tower by a swivel joint or any rotary joint or hinge such that the jib can rotate about the longitudinal axis of the tower 1211. For example, FIG. 12E shows a view of the crane 1210 with the jib 1212 rotated to position a hook 1214 of the crane 1210 above a component 1241. In an example implementation, the component 1241 includes a hook receiving element 1241A (e.g., a wire loop connected to the component 1241) to which the hook 1214 may be coupled for lifting the component 1241. FIG. 12F shows another view of the crane 1210 in the stowed configuration. The jib 1212 of the crane is rotated to form an angle a with the tower 1211, as described above. In an example implementation, the jib 1212 is configured to rotate about an axis 1219A of a connecting element 1219. The connecting element 1219 is configured to be attached to the tower 1211 (e.g., the connecting element 1219 is welded or bolted to the tower 1211), and is configured to support the jib 1212 in an operating configuration, as further shown in connection to FIG. 13B. In some variations, the connecting element 1219 may be rotatably mounted to the tower 1211 (e.g., coupled to a horizontal plate that is coupled to the tower via any rotary joint) so that the job 1212 may be rotated toward the drum (e.g., as depicted in FIG. 12E).

FIGS. 13A-13F show different views of the crane 1210. FIG. 13A shows a side view of the crane 1210 in a stowed configuration (similar to the view shown in FIG. 12F) with the jib 1212 rotated about axis 1219A of connecting element 1219. As previously shown, the base plate 1215 is configured to be connected to the support frame 1250 and forms an angle Q with the tower 1211. FIG. 13B shows the crane 1210 in the operating configuration in which the jib 1212 is positioned substantially perpendicular to the tower 1211 (and generally horizontal, i.e., perpendicular to a direction of gravity). In an example implementation, the jib 1212 may be secured in the horizontal position in the operating configuration via connecting element 1219 and a pin 1219B that can be inserted in a channel formed from aligned holes-a first hole in a side 1219C of the connecting element 1219 and a second hole in the jib 1212. In some cases, when the third hole in the side 1219D is present (the third hole is aligned with the first hole), the pin 1219B may be configured to penetrate through the entire channel formed by the first, the second and the third aligned holes. In an example implementation, the pin may be secured (e.g., with a bolt or any other suitable securing mechanism) from exiting the channel formed by the first, the second and the third aligned holes.

As further shown in FIG. 13B, tower 1211 includes a plate element 1211A solidly attached to a top end of tower 1211 (e.g., the plate element 1211A may be welded to the top end of the tower 1211), and an indentation 1211B in the plate element 1211A for the jib 1212 to enter when in a stowed configuration. That is, the jib 1212 may be retained in the indentation (or notch) 1211B when it is in the stowed configuration. Further, tower 1211 includes a lock sensor LS configured to detect when the jib 1212 is in the stowed configuration (e.g., the lock sensor LS may be a button for detecting a pressure resulting from the jib 1212, when the jib 1212 is inserted into the indentation 1211B and being placed in the stowed configuration). In some cases, the lock sensor LS is configured to communicate to a controller of the medical system that the jib 1212 is in a stowed configuration, and the controller may implement various control functions based on the data from the lock sensor LS. For example, the controller may be configured to prevent a rotation of a drum 1240 until the jib 1212 is in the stowed configuration. Optionally, the controller may be configured to inform a user (via any suitable means such as light signals or audio signals) that the jib 1212 is not in the stowed configuration. In one variation, placing the jib in the stowed configuration may trigger the generation of a signal in the lock sensor, and the signal may be communicated to the controller, which may generate command signals that permit the rotation of the drum (and optionally, generate a notification that is output to a display that indicates the crane is in its stowed configuration).

In some variations, system 1200 incudes a drum rotation mechanism capable of rotating the drum 1240. The lock mechanism LS is configured (e.g., via a suitable controller) to interface with the drum rotation mechanism, such that the lock mechanism LS is engaged when the crane 1210 is in the stowed configuration, and the drum rotation mechanism is configured to engage to rotate the drum 1240 when the lock mechanism is LS engaged. Further, the drum rotation mechanism is configured to stop a rotation of the drum 1240 when the lock mechanism LS is not engaged. In an example implementation, the lock mechanism LS includes a switch having a button, and wherein, when the crane 1210 is in the stowed configuration, an element of the jib 1212 is configured to depress the button resulting in the engagement of the drum rotation mechanism to rotate the drum.

FIG. 13B further shows a winch 1216 for winding in and out the hoist line 1213. In an example implementation, the hoist line 1213 is configured to pass through a cavity within the tower 1211 and the jib 1212. Thus, the hoist line 1213 may be entirely enclosed and managed internally within the crane 1210. In some variations, the winch 1216 is manually actuated, however, in other variations, the crane 1210 may have an electric drive, and the wires that provide power and control commands to the crane 1210 may be routed with the other wires associated with a support frame 1250 of the medical system 1200 and/or otherwise incorporated within a subsystem of the support frame 1250.

FIGS. 13C-13E further show details of the crane 1210, particularly details of the connecting element 1219 and an upper part of tower 1211. FIG. 13C shows the jib 1212 in a stowed configuration. As shown in FIG. 13C, the jib 1212 is secured in the stowed configuration by the same pin 1219A that secures it in the operating configuration. In the stowed configuration, the pin 1219A is configured to penetrate the first hole (H1) and the third hole (H3) while remaining outside of the jib 1212. A wire element (WE) is used to ensure that the pin 1219A does not get lost and may also be used to secure the exiting end EE of pin 1219A (e.g., when an end of the wire element WE is configured to couple to the exiting end EE).

FIG. 13D depicts the jib 1212 in an operational configuration (e.g., perpendicular to tower 1211). A lock sensor LS is configured to detect whether the jib 1212 is in the operational configuration or in the stowed configuration. The location of the lock sensor LS is such that the presence of the jib 1212 when it is in the stowed configuration is detectable (e.g., the jib contacts the lock sensor LS) and the absence of the jib 1212 when it is in the operational configuration is detectable (e.g., the jib no longer contacts the lock sensor LS). In the operational configuration, as shown in FIG. 13D, the notch 1211B is vacant and the lock sensor LS determines that the jib 1212 is in operational configuration. Conversely, when the jib 1212 is in the stowed configuration (as shown in FIG. 13E) a portion of the jib 1212 is located within the notch 1211B, and the lock sensor LS determines that the jib 1212 is in the stowed configuration. In some variations, a portion of the jib 1212 presses (or touches) the lock sensor LS. Alternatively, or additionally, the lock sensor LS may be configured to detect proximity of the jib 1212 (e.g., lock sensor LS may use optical means, such as laser and a photodiode to detect the proximity of the jib 1212). Alternatively, or additionally, the lock sensor may include a switch with a button, and when the jib is stowed, it depresses the button and generates a signal indicating that the crane is properly stowed, and rotation of the drum is permissible.

FIG. 13F shows a cross-sectional view of the crane 1210 with the jib 1212 being in an operational configuration. As shown, tower 1211 and jib 1212 are configured as a hollow cylindrical shells (e.g., rectangular cylindrical shells) having one or more internal grooves configured to retain and guide the hoist line 1213 within the tower 1211 and the jib 1212. The hoist line 1213 tension may be adjusted by a set of pulleys P1-P3. Further, the winding (and unwinding) of the hoist line 1213 is facilitated by a winch 1216. In an example implementation the winch 1216 may be geared (e.g., the winch 1216 may be a worm-gear winch). The winch 1216 may be, similar to or the same as, in form or in function, winch 316.

The examples and variations of cranes described herein have been provided in the context of maneuvering (e.g., removing and/or installing) components from a medical system, such as a radiotherapy system, however, it should be understood that the removable couch-mounted cranes and the permanent frame-mounted cranes described above may be used to maneuver components from any system comprising a bore. For example, the cranes and methods described herein may be used to maneuver components of a medical imaging system, such as a CT imaging system, and/or an MR imaging system, and the like. The couch-mounted cranes may be attached to any patient couch that has the appropriate coupling mechanisms, and the couch motion controls may be used to move the crane into a desired position within the system bore. The frame-mounted cranes may be attached to any support structure or chassis of a CT imaging system and/or an MR imaging system. Systems comprising a frame-mounted crane may include one or more of the lock mechanisms described above to ensure that the crane is in its stowed position before the system is operated.

While different variations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive aspects described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations and features described herein. It is, therefore, to be understood that the foregoing variations and features are presented by way of example only and that, within the scope of the appended claims and equivalents thereto; inventive variations may be practiced otherwise than as specifically described and claimed. Inventive variations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, variations may be constructed in which acts are performed in an order different than illustrated, which may include performing some acts simultaneously, even though shown as sequential acts in illustrative examples.

1. A removable crane for servicing a medical system, the removable crane configured to couple to a couch of the medical system, wherein upon securing the removable crane to the couch, a jib of the removable crane is positioned to enter a bore of a drum of the medical system upon a movement of the couch towards the drum.

2. The removable crane of claim 1, wherein the removable crane is configured to couple to the couch via a couch coupling mechanism attached to the couch.

3. The removable crane of claim 1, wherein upon securing the removable crane to the couch, the jib of the removable crane is substantially parallel to an axis of the bore.

4. The removable crane of claim 2, wherein the couch coupling mechanism comprises a clamp attached to a distal end of the couch.

5. The removable crane of claim 2, wherein the couch coupling mechanism comprises a left clamp attached to a left side of the couch and a right clamp attached to a right side of the couch.

6. The removable crane of claim 2, wherein the removable crane includes at least one extension element, and wherein the couch coupling mechanism comprises an opening configured to receive the extension element and latch to the extension element.

7. The removable crane of claim 2, wherein the couch coupling mechanism is configured to be attached at a distal end of the medical couch.

8. The removable crane of claim 2, wherein the couch coupling mechanism is configured to be attached at a position of the couch located between a substantially middle position of the couch and a distal end of the couch.

9. The removable crane of claim 2, wherein the removable crane is configured to execute at least one of a lateral or rotational motion relative to the couch.

10. The removable crane of claim 9, wherein the couch coupling mechanism includes a first element attached to the couch and a second element attached to the removable crane, and wherein the second element is configured to move laterally relative to the first element.

11. The removable crane of claim 10, wherein the first element includes one or more rails mounted on a platform configured to rotate in a plane parallel to a top surface of the couch, and the second element includes a set of wheels configured to move along the one or more rails.

12. The removable crane of claim 11, wherein an attachment of the removable crane to the second element allows the removable crane to rotate relative to the second element in a plane parallel to the top surface of the couch.

13. A removable crane installation system for servicing a medical system, wherein the medical system comprises:
a gantry;
a drum attached to a front face of a gantry, the drum having a bore; and
a couch capable of moving towards and away from the drum;
wherein the removable crane installation system comprises:
a removable crane configured to couple to the couch via a couch coupling mechanism, the removable crane having a tower and a jib connected to the tower, wherein, upon securing the tower to the couch, the jib is positioned to enter the opening of the drum upon a movement of the couch towards the drum.

14. The removable crane installation system of claim 13, wherein the medical system comprises one of a positron emission tomography (PET) device, and a computed tomography (CT) device.

15. The removable crane installation system of claim 13, wherein the tower is mounted substantially perpendicular to a top surface of the couch, and wherein the jib is positioned substantially perpendicular to the tower.

16. The removable crane installation system of claim 15, wherein drum includes components located inside the bore, and wherein the jib is configured to have a size that allows the jib to be inserted into the bore while lifting a component inside the bore, such that the component does not touch any sides of the bore.

17. The removable crane installation system of claim 13, wherein the couch coupling mechanism is attached to the couch via a left clamp attached to a left side of the couch and a right clamp attached to a right side of the couch.

18. The removable crane installation system of claim 13, wherein the couch coupling mechanism includes a groove, and the removable crane includes an extension element configured to slidably engage with the groove of the couch coupling mechanism.

19. The removable crane installation system of claim 13, wherein the couch coupling mechanism includes an opening, and the removable crane includes an extension element configured to slidably engage with the opening of the couch coupling mechanism.

20. A removable crane installation system for servicing a medical system, wherein the medical system comprises a couch, the couch including a couch patient platform comprising a platform base and a platform extension, wherein the platform extension is attached to the platform base via an attachment mechanism, the removable crane installation system comprising:
a removable crane, configured to attach to the attachment mechanism of the platform base.

21. The removable crane installation system of claim 20, wherein a jib of the removable crane is configured to be moved into a bore of a drum of the medical system to access a multileaf collimator (MLC) arc or a PET arc.

22. A medical system comprising:
a gantry; and
a crane permanently coupled to the gantry, the crane having a first configuration for use and a second stowed configuration.

23. The medical system of claim 22, wherein the crane includes a tower and a jib, and wherein in the second stowed configuration the jib of the crane folds from a substantially perpendicular direction to the tower to a direction that forms at most forty-five-degree angle with the tower.

24. The medical system of claim 23, further comprising a cover configured to cover the gantry, components of the imaging system, and the crane.

25. The medical system of claim 23, wherein the tower is mounted at a first angle to a surface of the gantry, and wherein an angle between the jib in the folded configuration and the tower is substantially the first angle, such that the jib is substantially parallel to a tangent direction to the surface of the gantry.

26. The medical system of claim 23, wherein the jib of the crane is configured to rotate about an axis passing through the tower of the crane.

27. The medical system of claim 23, wherein the gantry further comprises:
a drum rotatably coupled to a stationary frame of the gantry;
a component coupled to the drum, and
wherein the jib is coupled to the tower is by one of a hinge, a pivot, or a joint such that the jib is configured to rotate towards the drum and have an extension necessary to allow lifting the component.

28. The medical system of claim 23, further comprising a drum rotation mechanism capable of rotating the drum, and wherein the crane further includes a lock mechanism configured to interface with the drum rotation mechanism, such that the lock mechanism is engaged when the crane is in the second stowed configuration, and the drum rotation mechanism is configured to engage to rotate the drum when the lock mechanism is engaged and to stop a rotation of the drum when the lock mechanism is not engaged.

29. The medical system of claim 28, wherein the lock mechanism includes a switch having a button, and wherein, when the crane is in the second stowed configuration, an element of a crane is configured to depress the button resulting in the engagement of the drum rotation mechanism to rotate the drum.

30. A method for attaching a removable crane to a couch of a medical system, the method comprising:
installing a couch coupling mechanism onto the couch;
connecting a removable crane with the couch coupling mechanism,
wherein connecting the couch coupling mechanism and the removable crane includes:
sliding an extension element of the removable crane into an opening of the couch coupling mechanism.

31. The method of claim 30, wherein connecting the couch coupling mechanism and the extension element of the removable crane further includes:
positioning a tower of the removable crane at a first angle relative to a normal direction to a platform of the couch prior to the sliding of the extension element; and
positioning the tower of the removable crane at a second angle relative to the normal direction to the platform, after the sliding of the extension element, resulting in a completion of an installation of the removable crane; and
wherein a difference between the first and the second angle is at least five degrees.
